(19) United States
(12) Patent Application Publication
(10) Pub. No.: US 2022/0076827 A1
(43) Pub. Date: Mar. 10, 2022

(54) SYSTEMS AND METHODS FOR MODELLING A HUMAN SUBJECT

(71) Applicant: KONINKLIJKE PHILIPS N.V., EINDHOVEN (NL)

(72) Inventors: Cornelis Petrus Hendriks, Eindhoven (NL); Lieke Gertruda Elisabeth Cox, Eindhoven (NL); Murtaza Bulut, Eindhoven (NL); Valentina Lavezzo, Heeze (NL)

(21) Appl. No.: 17/016,545

(22) Filed: Sep. 10, 2020

(51) Int. Cl. G16H 50/20; G06N 20/00

(52) U.S. Cl. CPC G16H 50/20; G06F 2111/10; G06N 20/00

(57) ABSTRACT

There a proposed concepts for predicting the expiration date, or validity period, of a subject-specific digital twin. By calculating an expiration time of a digital twin model for a subject, proposed embodiments may enable an understanding of when digital twin predictions remain valid. In this way, it may be determined when the acquisition of data is desirable or required. Improved data collection may therefore be supported by proposed embodiments.

### FIELD OF THE INVENTION

The invention relates to modelling human subjects, and more particularly to the field of subject-specific models of biological function (commonly referred to as digital twins).

### BACKGROUND OF THE INVENTION

A recent development in healthcare is the so-called ‘digital twin’ concept. In this concept, a digital representation or computational simulation (i.e. the Digital Twin (DT)) of a physical system is provided and connected to its physical counterpart, for example through the Internet of things as explained in US 2017/286572 A1 for example. Through this connection, the DT model typically receives data pertaining to the state of the physical system, such as sensor readings or the like, based on which the DT model can predict the actual or future status of the physical system, e.g. through simulation.

Such DT technology is also becoming of interest in the medical field, as it provides an approach to more efficient medical care provision. For example, a DT model may be built using imaging data of a subject (i.e. patient), e.g. a person suffering from a diagnosed medical condition as captured in the imaging data.

The DT model(s) of a subject (i.e. subject-specific computational simulations) may serve a number of purposes. Firstly, the DT model(s) (rather than the patient) may be subjected to a number of virtual tests, e.g. treatment plans, to determine which treatment plan is most likely to be successful to the patient. This reduces the number of tests that physically need to be performed on the actual patient. Secondly, the DT(s) of a subject may be used to predict the onset, treatment (outcome) or development of medical conditions of the subject. That is, the DT model(s) of a subject may offer a healthcare professionals advanced visualization and/or physical insights into health information of the subject, thus supporting improved Clinical Decision Support (CDS).

A DT model is typically based on historical and new data. However, new data may not always be available and historical data may expire. For example, imaging data becomes invalid depending on how fast the medical, anatomical or physiological conditions change.

### SUMMARY OF THE INVENTION

The invention is defined by the claims.

According to examples in accordance with an aspect of the invention, there is provided a method for calculating an expiration time of a digital twin model for a subject after which the accuracy of the digital model does meet a required value, the method comprising:

obtaining a disease state of subject at a first point in time and a tolerance value representative of a threshold in the disease state of the subject;

determining a modification rate of the disease state for the subject based on the digital twin model for the subject;

determine a value of uncertainty of the modification rate; and

calculating an expiration time of the digital twin model for the subject based on: the first point in time; the disease state of the subject; the tolerance value; the determined modification rate; and the determined value of uncertainty of the modification rate.

Embodiments propose concepts for predicting the expiration date, or validity period, of a subject-specific DT model. A result provided by such a prediction may comprise an “intelligent timestamp”. Such a timestamp may be considered ‘intelligent’ because it is representative of a predicted future date. It may also be dynamic, i.e. it may continuously change or be updated when new data becomes available (e.g. medical data (scan), wearables data (weight) or lifestyle data (smoking)), medication usage. Also, when the DT model is updated, the timestamp may be updated. That is, the predicted expiry date may be based on the subject-specific DT model.

By calculating an expiration time of a DT model for a subject, proposed embodiments may enable an understanding of when DT model predictions remain valid. In this way, it may be determined when the acquisition of data is desirable or required. Improved data collection may therefore be supported by proposed embodiments.

Embodiments may therefore be of particular use for supporting clinical decision making. Exemplary usage applications may for example, relate to predicting the onset, treatment (outcome) or development of medical conditions and/or medical procedures. Embodiments may thus be of particular use in relation to medical care management and/or prediction.

For example, potential benefits from the proposed concept(s) for predicting the expiration date of a subject-specific DT model may include: improved accuracy and/or reliability of medical outcome predictions; reduced costs via improved medical treatment planning; improved subject (i.e. patient) satisfaction (e.g. reduction in unnecessary scans and/or medical care visits; and improved staff satisfaction (e.g. via improved decision support and reduced uncertainty).

The threshold in the disease state of the subject may be representative of: a target disease state; a next disease state of the subject; a maximum acceptable disease state of the subject; a maximum acceptable modification to the disease state of the subject. Embodiments may therefore cater for various approaches to defining a threshold. In this way, embodiments may be adapted to clinical requirements and/or preferences.

In an embodiment, obtaining a tolerance value may comprise determining the tolerance value based on at least one of: a physiological property of the subject; and medical guidelines. In this way, embodiments may make use of various forms of information to determine a subject-specific tolerance value that is better-suited to the subject.

Determining a modification rate of the disease state for the subject may be further based on at least one of: historical data relating to a medical history of the subject; clinical knowledge or a similar patient algorithm; a subject-specific disease progression model. Embodiments may therefore leverage various forms of information to model subject-specific disease progression and thus improve prediction accuracy.

In some embodiments, calculating an expiration time of the DT model for the subject may comprise determining an expiration time t2 according to the following equation: t2=t1+(T−X−δ/2)/tamp, wherein: tl is the first point in time; T is the tolerance value; X is the disease state of the subject at t1; φ is the modification rate; and δ is the value of uncertainty of the modification rate. Relatively simple mathematical operations and calculations may there be employed by embodiments, thus minimizing the cost and/or complexity associated with implementation.

Some embodiments may further comprise obtaining a second disease state of the subject at a second point in time after the first point in time. An accuracy of the determined modification rate may then be determined based on the second disease state. Further, the DT model for the subject may be modified based on the second disease state. Feedback concepts may thus be employed for improved accuracy and/or continued improvement of embodiments.

Embodiments may further comprise generating a timestamp for the DT model for the subject based on the calculated expiration time. The generated timestamp may be configured to be computer-readable, thus enabling automated use by a computer system (e.g. for display and/or control purposes).

In an embodiment, the processor arrangement may be further configured to generate a control instruction for a user interface medical equipment based on the calculated expiration time of the DT model. In this way, a user interface may display information and/or warnings. As a further example, medical equipment may controlled (e.g. new tests scheduled) according to results (e.g. predictions) generated by an embodiment. Dynamic and/or automated control concepts may therefore be realized by proposed embodiments.

According to examples in accordance with yet another aspect of the invention, there is provided a computer program product comprising computer program code means which, when executed on a computing device having a processing system, cause the processing system to perform all of the steps of the method described above

According to examples in accordance with another aspect of the invention, there is provided a system for calculating an expiration time of a DT model for a subject after which the accuracy of the digital model does meet a required value, the system comprising:

an input interface configured to obtain a disease state of subject at a first point in time and a tolerance value representative of a threshold in the disease state of the subject;

a model analysis component configured to determine a modification rate of the disease state for the subject based on the DT model for the subject and to determine a value of uncertainty of the modification rate; and

a processor unit configured to calculate an expiration time of the DT model for the subject based on: the first point in time; the disease state of the subject; the tolerance value; the determined modification rate; and the determined value of uncertainty of the modification rate.

Further, proposed concepts may provide a clinical decision support comprising a system according to a proposed embodiment.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a graph depicting a concept method for calculating an expiration time of a DT model for a subject according to the invention;

FIG. 2 depicts a flow diagram of a method for calculating an expiration time of a DT model for a subject according to an exemplary embodiment;

FIG. 3 is a simplified block diagram of a system for calculating an expiration time of a DT model for a subject according to an embodiment; and

FIG. 4 illustrates an example of a computer within which one or more parts of an embodiment may be employed.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for predicting when an accuracy of a DT model for a subject may no longer be valid. For instance, proposed embodiments may provide an approach to calculating a point of time in the future after which the accuracy of a subject-subject DT model does not meet a required value. By considering the modification rate of a disease state for the subject, a value of uncertainty of the modification rate, and a threshold disease state, embodiments can calculate an earliest point in time at which the threshold disease state may be reached. That is, accounting for the uncertainty in the modification rate (i.e. rate of progression) of the disease as modelled by the DT model for the subject, embodiments may identify a point in time at which the disease state of the subject may progress to the threshold disease state. Such a point in time may, for example, predict an earliest time at which further action with respect to the subject may be preferable to undertake. The predicted point in time (i.e. expiration time) may be used to generate a timestamp for the DT model.

Proposed embodiments may therefore leverage the benefits of both a subject-specific DT model and relatively simple mathematical prediction techniques.

Reference to a ‘timestamp’ should be taken to refer to a sequence of characters or encoded information identifying the time of occurrence of an event (which may be in the past or in the future). A timestamp thus identifies when a certain event occurs, usually by specifying a date and time, sometimes accurate to a small fraction of a second. The term derives from rubber stamps used in offices to stamp the current date to record when the document was received. In modern times, usage of the term has expanded to refer to digital date and time information attached to digital data. For example, computer files contain timestamps that identify when the file was last modified, and digital cameras add timestamps to the captured images, recording the date and time the image was captured.

It is known to use timestamps to monitor clinical workflows, and based on the timestamp information, improvement in a clinical workflow may be determined. Also, in current clinical practice, new data acquisitions are planned in guidelines, e.g. screening intervals based on initial findings. For example, based on an actual disease stage of a subject, a statistical model may be used to prescribe the time of a next visit to doctor. The model may for example be designed to reduce costs (e.g. via reducing unwarranted/unnecessary visits) or to reduce a risk that a subject reaches a disease stage which requires medical intervention. Proposed embodiments may enable more accurate modeling of the disease progression and/or future disease state of a subject and their need for imminent medical intervention. Embodiments may therefore be of particular use for supporting clinical decision-making.

Exemplary usage applications may, for example, relate to predicting the onset, treatment (outcome) or development of medical conditions and/or medical procedures.

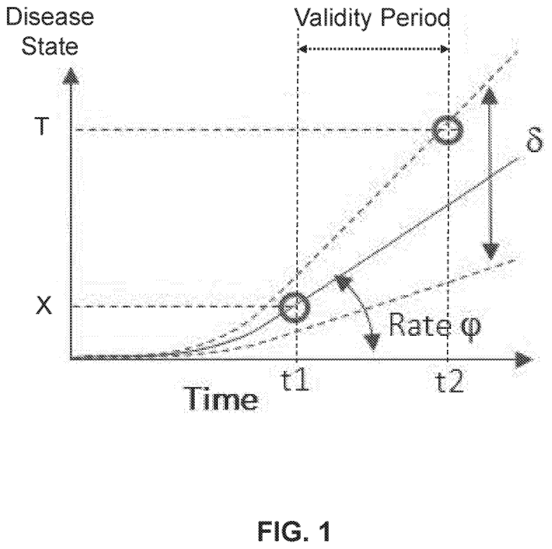

Referring now to FIG. 1, there is illustrated a graph depicting a concept according to the invention. The graph depicts disease state (y-axis) against time (x-axis).

The disease stage X of a person at a moment in time, t1, is obtained from medical records or determined by a medical text/examination.

A tolerance value T is then determined according a target disease state of the subject. This may be the disease state that can be reached without requiring any further medical action (e.g. test or treatment). For instance, the tolerance value T may be representative of a next disease stage, or a certain amount of acceptable disease progression (i.e. deterioration) relative to disease state X (e.g. 50% deterioration).

A disease modification (i.e. progression) rate φ is then determined according to DT model for the subject which models disease progression based on subject historical data, clinical knowledge and/or similar subject-specific algorithms. As can be seen from FIG. 1, the disease modification (i.e. progression) rate φ is representative of the rate of change of the disease state (i.e. disease progression) with respect to elapsed time, and so may be considered as the gradient of the line representing variation/progression of the disease state against time.

The uncertainty δ of the predicted disease modification rate is also obtained. This may, for example, be obtained using a variance analysis (e.g. variation in DT model output depending on the variation in input and DT model parameters). As can be seen from FIG. 1, the uncertainty δ value may define a maximum and minimum predicted disease state at a particular time. Thus, a higher uncertainty δ value will result in the maximum and minimum predicted disease state at a particular point in time being further away from the predicted disease at a point in time as predicted by the DT model for the subject. The uncertainty δ is therefore expected to increase with time elapsed since a known/measured disease state.

According to simple geometric calculations, the expiration time t2 of the DT model (e.g. the earliest predicted time by which the tolerance value T may be met) can then be calculated according to Equation I as follows:

t2=t1+(T−X−δ/2/tan φ (I).

Although the above approach employs a simple linear progression model, it will be appreciated that more complex non-linear progression models may be employed in other embodiments. Thus, the exemplary concept illustrated with reference to FIG. 1 is simply provided to demonstrate the underlying concept(s) proposed and the various parameters that may be employed.

The expiration time t2 can be recalculated whenever new input data for the DT model becomes available. For example, new data may become available that indicates the actual disease stage is different from a predicted disease stage, thus enabling the disease modification rate φ to be recalculated and used for a new prediction.

The predicted expiration time t2 may be used to define an intelligent timestamp that can be used in different ways.

For example, when using a subject-specific DT model, the timestamp can be used to indicate a validity period of the model (e.g. via a user interface). This may be done using a simple binary indication (e.g. valid, not valid). Of course, more detailed information and instructions regarding the predicted expiration time t2 may be provided, e.g. a measure of reliability of predictions with respect to time, such as ‘90% reliability after one month’, ‘50% reliability after three months’, etc.

According to another example, when nearing or reaching the expiration date, a warning may be provided and/or previous disease prediction invalidated (e.g. via a signal or flag in a user interface). This may be used to prompt collection of new data.

In yet another example, the calculated an expiration time may be used to plan and/or recommend the data and time of a new medical examination/test.

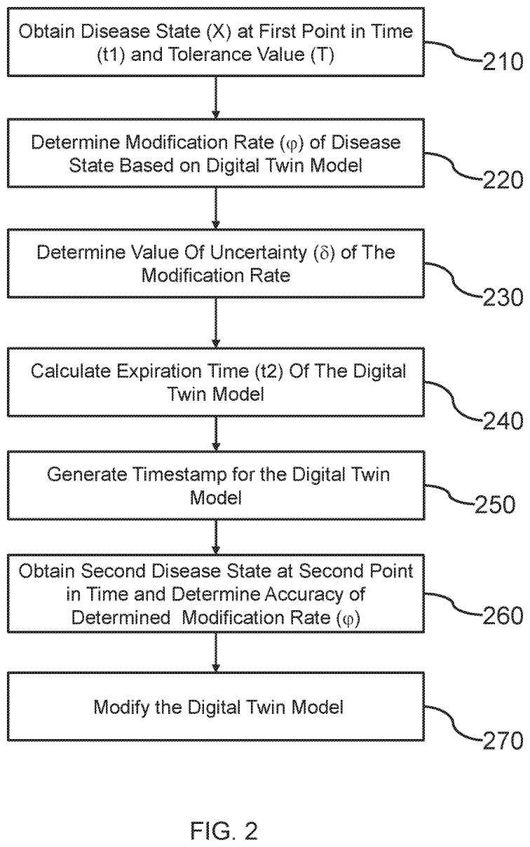

Referring now to FIG. 2, there is depicted a flow diagram of a method for calculating an expiration time of a DT model for a subject according to an exemplary embodiment.

The method begins with step 210 of obtaining a disease state X of subject at a first point in time t1 and a tolerance value T representative of a threshold in the disease state of the subject. In this example, the threshold in the disease state of the subject is representative of a next disease state of the subject. It can, however, be representative of threshold values such as: a target disease state; a maximum acceptable disease state of the subject; or a maximum acceptable modification to the disease state of the subject. Also, it is noted that the tolerance value is determined based on at least one of: a physiological property of the subject; and medical guidelines. In this way, the tolerance value is adapted to cater for subject-specific factors and medical guidelines relating to those factors.

In step 220, a modification rate φ of the disease state for the subject is determined according to a DT model for the subject. In doing so, determining the modification rate of the disease state for the subject is further based on at least one of: historical data relating to a medical history of the subject; clinical knowledge or a similar patient algorithm; a subject-specific disease progression model.

In step 230, a value of uncertainty δ of the modification rate is determined. In this example, the value of uncertainty δ is determined using variance analysis which takes account of variation in DT model outputs according to variations in inputs and model parameters.

In step 240, an expiration time t2 of the DT model for the subject is calculated based on: the first point in time; the disease state of the subject; the tolerance value; the determined modification rate; and the determined value of uncertainty of the modification rate. Specifically, in this example, the disease progression is modelled as a linear function and so the expiration time t2 is calculated according to Equation I (detailed above).

A timestamp for the DT model is then generated in step 250 based on the calculated expiration time.

Further, in the method of FIG. 1, the method comprises the additional steps of 260 and 270. Step 260 comprises obtaining a second disease state of the subject at a second point in time after the first point in time, and then determining an accuracy of the determined modification rate based on the second disease state. The accuracy may for example be determined by simply comparing the second disease state with a predicted disease state at the second point in time according to the DT model. Based on the second disease state (or the accuracy of the determined modification rate), the DT model is modified. In this way, the availability of new data that indicates an actual disease stage differs from a predicted disease stage can be used to refine the DT model.

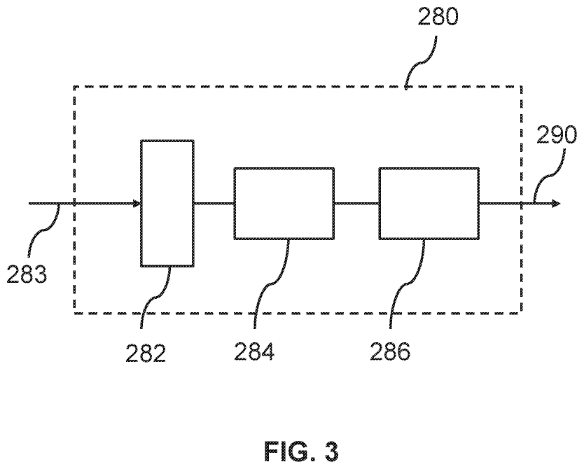

Referring now to FIG. 3, there is depicted a simplified block diagram of a system 280 for calculating an expiration time of a DT model for a subject according to an embodiment.

The system comprises an input interface 282 that is configured to obtain an input data signal 283. The input data signal 283 comprises information relating to a measured disease state of subject at a first point in time and a tolerance value representative of a threshold in the disease state of the subject. In this example, the tolerance value represents a maximum acceptable disease state of the subject.

A model analysis component 284 of the system 280 is configured to determine a modification rate of the disease state for the subject based on a DT model for the subject and to determine a value of uncertainty of the modification rate.

A processor unit 286 of the system 280 is configured to calculate an expiration time of the DT model for the subject based on the information acquired or determined by the input interface 282 and model analysis component 284. That is, the processor unit 286 calculates an expiration time of the DT model based on: the first point in time; the disease state of the subject; the tolerance value; the determined modification rate; and the determined value of uncertainty of the modification rate.

As described above, if the disease progression with respect to time is modelled as being generally linear, the processor unit 286 may employ Equation I to calculate an expiration time of the DT model. However, where the disease progression with respect to time is modelled as being non-linear, the processor unit 286 may employ alternative calculations (and/or assumptions) to calculate an expiration time of the DT model. Such calculations (and/or assumptions) will be within the general knowledge and/or capabilities of a person skilled in the art.

The processor unit 286 is also configured to generate and output a timestamp 290 for the DT model for the subject based on the calculated expiration time.

To provide further understanding of the potential applications of the proposed concept(s), some exemplary use cases will now be detailed in view of disease prediction models that are known and available in published literature.
Atherosclerosis is a disease in which arteries slowly narrow due to the build of plaque. This can lead to a sudden stroke (when the plaque ruptures), or to chronic problems such as peripheral artery disease. The build-up of plaque can be calculated with linear plaque build-up models (Liu B, Tang D. Computer simulations of atherosclerotic plaque growth in coronary arteries. Mol Cell Biomech. 2010;7(4):193-202.). The disease stage X of a person at a moment in time, t1, can be determined with ultrasound (for example, an artery diameter at a location where the intima media thickness, i.e. the thickness of the two innermost layers of the artery wall shows an abnormal thickening. The tolerance value T can be an arbitrary disease stage, for example a 10% decrease in predicted artery diameter compared to the initial diameter, at which a new ultrasound scan can be planned to monitor the disease and/or update the model. Alternatively, when there is a high level of certainty in the model predictions, the tolerance value T can be the disease stage that requires an intervention, e.g. stent placement, balloon angioplasty or arterial bypass surgery. In this case, T can be for example 75% reduction in vessel diameter.
Abdominal aortic aneurysm (AAA) is a disease in which the abdominal aortic artery exhibits a (growing) dilatation. This dilatation is detected and diagnosed with ultrasound, CT or angiography. At some point the artery can rupture with fatal consequences. Lee R, Jarchi D, Perera R, et al. (Applied Machine Learning for the Prediction of Growth of Abdominal Aortic Aneurysm in Humans. EJVES Short Rep. 2018;39:24-28. Published 2018 May 1. doi:10.1016/j.ejvssr.2018.03.004) provides a machine learning model to predict annual AAA growth for individual patients based on their initial AAA diameter. The tolerance value T can be an arbitrary disease stage (for example, a 10% increase in predicted AAA diameter compared to the initial diameter) at which a new (ultrasound) scan can be planned to monitor the disease and/or the model can be updated. Alternatively, when there is a high level of certainty in the model predictions, the tolerance value T can be the disease stage which requires an intervention, e.g. stent graft placement or surgery. In this case, the tolerance value T can be for example an artery diameter AAA=5 cm.
Emphysema is a lung disease in which the alveoli are progressively and irreversibly damaged causing a shortness of breath. Smoking is a main risk factor for Emphysema. Ceresa et al. (Ceresa M, Olivares A L, Noailly J, Gonzalez Ballester M A. Coupled Immunological and Biomechanical Model of Emphysema Progression. Front Physiol. 2018;9:388. Published 2018 Apr. 19.) describe a multiscale model to simulate the onset and progression of emphysema based on CT images and smoking behaviour. The model output is cell death and mechanical damage. The tolerance value T can be a next disease stage or an end stage, depending on the clinical objective.
Lorenzo et al. (Lorenzo-Redondo R, Fryer H R, Bedford T, et al. Persistent HIV-1 replication maintains the tissue reservoir during therapy. Nature. 2016;530(7588):51-56) describe a tissue scale personalized computer simulation of prostate tumor growth based on the anatomy extracted from medical images. The model output is the tumor geometry and volume. A DT of the prostate with the tumor inside might support the active surveillance of early stage PCa complementary to PSA blood tests. The tolerance value T may be defined to be an increase in volume of X% or a next clinical disease stage.

In the above examples, the disease stage is characterized by a single number. However, embodiments need not be limited to one-dimensional functions but instead may also be applied in multi-dimensional staging systems, such as TNM cancer staging.

Also, for implementations where the DT model has multiple outputs, (e.g. a heart, a lung and a diabetes parameter), an expiration time (and associated timestamp) can be for each per output (rather than a single expiration time or timestamp).

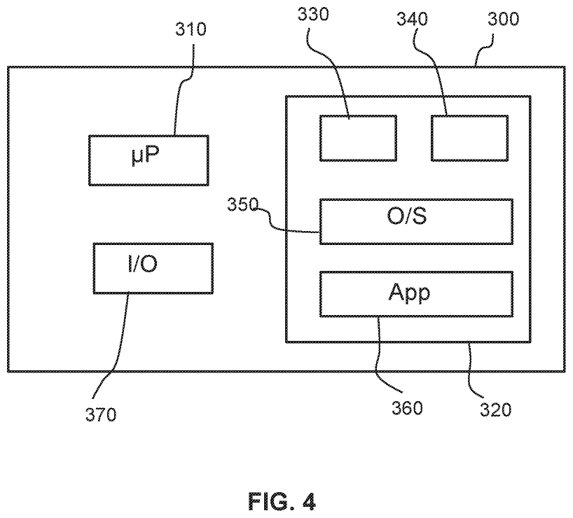

By way of further example, FIG. 4 illustrates an example of a computer 300 within which one or more parts of an embodiment may be employed. Various operations discussed above may utilize the capabilities of the computer 300. For example, one or more parts of a system for calculating an expiration time of a DT model for a subject may be incorporated in any element, module, application, and/or component discussed herein. In this regard, it is to be understood that system functional blocks can run on a single computer or may be distributed over several computers and locations (e.g. connected via internet).

The computer 300 includes, but is not limited to, PCs, workstations, laptops, PDAs, palm devices, servers, storages, and the like. Generally, in terms of hardware architecture, the computer 300 may include one or more processors 310, memory 320, and one or more I/O devices 370 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 310 is a hardware device for executing software that can be stored in the memory 320. The processor 310 can be virtually any custom made or commercially available processor, a central processing unit (CPU), a digital signal processor (DSP), or an auxiliary processor among several processors associated with the computer 300, and the processor 310 may be a semiconductor based microprocessor (in the form of a microchip) or a microprocessor.

The memory 320 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 320 may incorporate electronic, magnetic, optical, and/or other types of storage media. Note that the memory 320 can have a distributed architecture, where various components are situated remote from one another, but can be accessed by the processor 310.

The software in the memory 320 may include one or more separate programs, each of which comprises an ordered listing of executable instructions for implementing logical functions. The software in the memory 320 includes a suitable operating system (O/S) 350, compiler 340, source code 330, and one or more applications 360 in accordance with exemplary embodiments. As illustrated, the application 360 comprises numerous functional components for implementing the features and operations of the exemplary embodiments. The application 360 of the computer 300 may represent various applications, computational units, logic, functional units, processes, operations, virtual entities, and/or modules in accordance with exemplary embodiments, but the application 360 is not meant to be a limitation.

The operating system 350 controls the execution of other computer programs, and provides scheduling, input-output control, file and data management, memory management, and communication control and related services. It is contemplated by the inventors that the application 360 for implementing exemplary embodiments may be applicable on all commercially available operating systems.

Application 360 may be a source program, executable program (object code), script, or any other entity comprising a set of instructions to be performed. When a source program, then the program is usually translated via a compiler (such as the compiler 340), assembler, interpreter, or the like, which may or may not be included within the memory 320, so as to operate properly in connection with the O/S 350. Furthermore, the application 360 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 370 may include input devices such as, for example but not limited to, a mouse, keyboard, scanner, microphone, camera, etc. Furthermore, the I/O devices 370 may also include output devices, for example but not limited to a printer, display, etc. Finally, the I/O devices 370 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 370 also include components for communicating over various networks, such as the Internet or intranet.

If the computer 300 is a PC, workstation, intelligent device or the like, the software in the memory 320 may further include a basic input output system (BIOS) (omitted for simplicity). The BIOS is a set of essential software routines that initialize and test hardware at startup, start the O/S 350, and support the transfer of data among the hardware devices. The BIOS is stored in some type of read-only-memory, such as ROM, PROM, EPROM, EEPROM or the like, so that the BIOS can be executed when the computer 300 is activated.

When the computer 300 is in operation, the processor 310 is configured to execute software stored within the memory 320, to communicate data to and from the memory 320, and to generally control operations of the computer 300 pursuant to the software. The application 360 and the O/S 350 are read, in whole or in part, by the processor 310, perhaps buffered within the processor 310, and then executed.

When the application 360 is implemented in software it should be noted that the application 360 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 360 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

A single processor or other unit may fulfill the functions of several items recited in the claims.

It will be understood that the disclosed methods are computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A method for calculating an expiration time of a digital twin model for a subject after which the accuracy of the digital model does meet a required value, the method comprising:
obtaining a disease state (X) of subject at a first point in time (t1) and a tolerance value (T) representative of a threshold in the disease state of the subject;
determining a modification rate (φ) of the disease state for the subject based on the digital twin model for the subject;
determine a value of uncertainty (δ) of the modification rate; and
calculating an expiration time (t2) of the digital twin model for the subject based on: the first point in time; the disease state of the subject; the tolerance value; the determined modification rate; and the determined value of uncertainty of the modification rate.

2. The method of claim 1, wherein the tolerance (T) in the disease state of the subject is representative of: a target disease state; a next disease state of the subject; a maximum acceptable disease state of the subject; a maximum acceptable modification to the disease state of the subject.

3. The method of claim 1, wherein obtaining a tolerance value (T) comprises:
determining the tolerance value based on at least one of: a physiological property of the subject; and medical guidelines.

4. The method of claim 1, wherein determining a modification rate (φ) of the disease state for the subject is further based on at least one of: historical data relating to a medical history of the subject; clinical knowledge or a similar patient algorithm; a subject-specific disease progression model.

5. The method of claim 1, wherein calculating an expiration time (t2) of the digital twin model for the subject comprises:
determining an expiration time (t2) according to the following equation:
t2=t1+(T−X−δ/2)/tan φ
wherein: t1 is the first point in time; T is the tolerance value; X is the disease state of the subject at t1; φ is the modification rate; and δ is the value of uncertainty of the modification rate.

6. The method of claim 1, further comprising:
obtaining a second disease state of the subject at a second point in time after the first point in time,
and determining an accuracy of the determined modification rate based on the second disease state.

7. The method of claim 6, further comprising:
modifying the digital twin model for the subject based on at least one of the determined accuracy and the second disease state.

8. The method of claim 1, further comprising:
generating a timestamp for the digital twin model for the subject based on the calculated expiration time.

9. A computer program product comprising computer program code means which, when executed on a computing device having a processing system, cause the processing system to perform all of the steps of the method according to claim 1.

10. A system for calculating an expiration time of a digital twin model for a subject after which the accuracy of the digital model does meet a required value, the system comprising:
an input interface configured to obtain a disease state (X) of subject at a first point in time (t1) and a tolerance value (T) representative of a threshold in the disease state of the subject;
a model analysis component configured to determine a modification rate (φ) of the disease state for the subject based on the digital twin model for the subject and to determine a value of uncertainty (δ) of the modification rate; and
a processor unit configured to calculate an expiration time (t2) of the digital twin model for the subject based on: the first point in time; the disease state of the subject; the tolerance value; the determined modification rate; and the determined value of uncertainty of the modification rate.

11. The system of claim 10, wherein the threshold in the disease state of the subject is representative of: a target disease state; a next disease state of the subject; a maximum acceptable disease state of the subject; a maximum acceptable modification to the disease state of the subject.

12. The system of claim 10, wherein the input interface is configured to determine the tolerance value based on at least one of: a physiological property of the subject; and medical guidelines.

13. The system of claim 10, wherein the processing unit is configured to determine an expiration time t2 according to the following equation:
t2=t1+(T−x−δ/2)/tan φ
wherein: t1 is the first point in time; T is the tolerance value; X is the disease state of the subject at t1; φ is the modification rate; and δ is the value of uncertainty of the modification rate.

14. The system of claim 10, wherein the processor is configured to generate a timestamp for the digital twin model for the subject based on the calculated expiration time.

15. A clinical decision support comprising a system for calculating an expiration time of a digital twin model for a subject according to claim 10.
